(19) United States
(12) Patent Application Publication
(10) Pub. No.: US 2013/0052145 A1
(43) Pub. Date: Feb. 28, 2013

(54) ADHESIVE SLOW-RELEASE FORMULATIONS FOR THE LOCAL ADMINISTRATION OF CURCUMIN

(71) Applicant: Andreas Obwaller, Vienna (AT)

(72) Inventor: Andreas Obwaller, Vienna (AT)

(73) Assignee: Orphanidis Pharma Research GmbH, Vienna (AT)

(21) Appl. No.: 13/642,142

(22) PCT Filed: Apr. 29, 2011
(86) PCT No.: PCT/EP2011/056836

§ 371 Date: Nov. 7, 2012

(30) Foreign Application Priority Data
Apr. 30, 2010 (AT) A 737/2010

(51) Int. Cl. A61K 31/12; A61Q 11/00; A61K 8/86; A61P 29/00; A61P 35/00; A61K 8/81; A61P 31/00

(52) U.S. Cl. 424/49; 514/679

(57) ABSTRACT

Through the combined use of solvents with polyacrylic acid, it is possible to produce curcumin formulations that are stable in liquid form, while having both mucoadhesive and slow-release properties and ensure a solubility of the active ingredient of up to 15% (w/v).

Curcumin is a compound with an intense orange-yellow colour; it occurs in natural form in turmeric, also known as Curcuma longa. New studies have shown that when curcumin is applied topically, it has anti-inflammatory and antimicrobial effects. It is necessary here to keep the active ingredient in contact with the tissue to be treated as long as possible.

Reaching this goal is a major pharmaceutical technological challenge and a problem that has not yet been solved through formulation technology. The reason for this is mainly that in liquid and semisolid preparations, adhesion of the active ingredient to the mucous membranes, such as the oral, vaginal or rectal mucosa and/or to the skin must be prolonged, while on the other hand, the active ingredient must be released slowly in order to achieve the desired effect. However, solid dosage forms in which properties such as adhesion and slow release are already the state of the art are out of the question for achieving the largest possible areas in contact with the active ingredient. In addition, there is the problem that curcumin itself does not have a charge, which would facilitate a slow-release formulation of the active ingredient with charged carrier polymers because of ionic interactions. Another problem is that vehicle systems having adhesive properties on mucous membranes lose these mucoadhesive properties when an active ingredient is bound to them at the same time, but this is essential for a slow-release formulation. Furthermore, curcumin is sparingly soluble in an aqueous medium.

WO 2006/027248 A2 describes the use of a variety of different plant extracts, including those from the Zingiberales order, for inhibiting the dextran sucrase.

A method for producing a curcuma plant extract and a composition for cosmetic applications containing an extract of curcuma plants is described in EP 2 057 995 A2.

WO 2010/070664 A1 describes the use of curcuminoids such as curcumin for treatment of eye diseases.

WO 2010/115852 A1 describes the use of curcuminoids in combination with docetaxel for treatment of cancer, where the curcumin is preferably administered orally.

The object of the present invention is therefore to make available a formulation in which curcumin is in a stable solution and preferably has mucoadhesive and slow-release properties.

The object of the invention is achieved by a composition containing curcumin, polyacrylic acid, a solvent and optionally other additives.

In the invention disclosed here, a method has surprisingly been found for combining both properties—adhesion and slow release—in one liquid formulation and keeping curcumin in solution even in high concentrations. It has been found that polyacrylic acid, which is known as an adhesive polymer and in particular a mucoadhesive polymer in combination with a solvent is capable of ensuring a slow release of the active ingredient even in liquid preparations without any loss of the adhesive power (V. Grabovac, D. Guggi, A. Bernkop-Schnürch, Comparison of the mucoadhesive properties of various polymers, Adv Drug Deliv Rev. 2005 Nov. 3; 57(11):1713-23).

According to a special embodiment of the invention, curcumin may be present in the composition in a concentration of 0.1% to 15%, preferably from 1% to 10%.

The solvent may be selected in particular from the group consisting of dimethyl-acetamides, polyethoxyated oleic acid glycerides (Labrafil), pyrrolidones, N-ethyl-2-pyrrolidones, N-methyl-2-pyrrolidones, polyethylene glycols or polyoxamers such as methyloxirane (Pluronic L44). Polyethylene glycol having a molecular weight of 106 to 10,000 Da, preferably 300 to 6000 Da, may preferably be used.

Polyacrylic acid in particular has a molecular weight of 1 to 10,000 kDa and may optionally be crosslinked.

The composition may contain additional excipients, for example, but not limited to water, neutralizing agents, thickening agents, tastes correctors, dyes, preservatives and stabilizers.

According to the present invention, the composition may be in any form that may be suitable for use with curcumin, preferably a liquid or gel formulation, such as a cream, an ointment or a rinse. Alternatively, the formulation according to the invention may also be in the form of a solid formulation, for example, as a suppository, preferably as a vaginal suppository. Curcumin may be formulated as a slow-release liquid formulation for example.

Through the composition according to the invention, an at least 10%, preferably at least 20% slow-release effect of curcumin in the liquid form of the formulation within 30 minutes is achieved. The invention also includes a pharmaceutical preparation containing the formulation according to the invention and its therapeutic use. The invention also includes the use of the composition in combination with an analgesic.

### FIGURES

FIG. 1: Release of curcumin from a PEG 300 solution with various concentrations of crosslinked polyacrylic acid (Carbopol 974P NF) is shown (grey: 0% polyacrylic acid; black: 0.05% polyacrylic acid; grey hatching: 0.1% polyacrylic acid; black hatching: 0.5% polyacrylic acid). The values shown here correspond to the average of at least three experiments each (±standard deviation).

FIG. 2: Graphic plot of the mucoadhesive properties of pure crosslinked polyacrylic acid (Carbopol 974P NF) and crosslinked polyacrylic acid with 2% curcumin. The values shown here correspond to the average of at least three experiments each (±standard deviation).

FIG. 3: HPLC spectrum before (top) and after (bottom) one month of storage.

FIG. 4: Ointment number 1 produced from 2% curcumin, 12.5% PEG 6000, 19% PEG 600 and 66.5% PEG 300 was stable in storage for 4 weeks at 40° C./75% relative atmospheric humidity. The values shown here correspond to the average of at least three experiments each (±standard deviation).

The invention comprises a composition containing curcumin, polyacrylic acid and a solvent in which curcumin is soluble such that this composition advantageously has a slow release of active ingredient.

The term “curcumin” according to the present invention includes curcumin and metabolites or analogs of curcumin under the assumption that the metabolites or analogs have an anti-inflammatory or antimicrobial effect. Examples of metabolites include dihydroferulic acid, ferulic acid, glycocides of tetrahydrocurcumin or hydroxyhydrocurcumin.

Curcumin may be obtained as an isolate from the natural source Curcuma longa L. or may be synthesized chemically. Curcumin also includes isomers of curcumin, pharmaceutically acceptable salts thereof, precursors of curcumin or polymorphs or tautomers thereof. Curcumin may also be formulated as a metal chelate, for example, as a copper chelate.

The preferred release of active ingredient could be based on the development of hydrogen bridge bonds between the phenolic partial structures of curcumin with the carboxylic acid partial structures of polyacrylic acid which are stabilized by the presence of a solvent, for example, polyethylene glycol (PEG).

It has surprisingly been demonstrated that although solvents such as PEGs are normally incompatible with phenolic compounds in higher concentrations, this effect does not occur in the combined use of polyacrylic acid with solvents such as PEG even at high concentrations.

Despite the binding of curcumin to polyacrylic acid, it has been found that the polymer surprisingly does not thereby lose its adhesive properties. The formulation according to the invention therefore also has adhesive properties on body surfaces and/or skin surfaces, preferably mucoadhesive properties, for example, on oral mucosa, vaginal or rectal mucosa. These adhesive properties are also especially advantageous for the slow release of the active ingredient on the surface to which the formulation is applied. Therefore the active ingredient can adhere to the skin surface for a longer period of time, be released through the skin surface and can also manifest its efficacy for a longer period of time.

The formulation according to the invention preferably also has a high stability in storage. Curcumin, which is normally very unstable in liquid preparations because of its oxidation susceptibility, surprisingly has a high stability in storage in the formulations according to the invention.

The curcumin content can be selected according to the therapeutic requirements of the composition. According to a special embodiment of the invention, curcumin may also be present in higher concentrations in the composition in a stable form, for example, in a concentration up to 2.5% (w/v).

Curcumin is preferably present in a concentration of 0.1% to 15%, preferably 1% to 10%.

According to the present invention, any solvent in which curcumin can be dissolved and in which curcumin has a high enough stability in storage may be selected.

According to the present invention, the “solvent” may be understood to be any mixture or chemical compound which improves the solubility of curcumin under physiological conditions. Physiological conditions are understood to refer to a pH range of 4-8 and a temperature of 30-42° C.

A solubility of at least 0.5%, preferably at least 1% is preferred. The solubility of curcumin can easily be tested by those skilled in the art by using known methods of determining solubility.

The solvent is preferably selected from the group consisting of dimethylacetamide, polyethoxyated oleic acid glycerides (Labrafil), pyrrolidone, N-ethyl-2-pyrrolidone, N-methyl-2-pyrrolidone, polyethylene glycol, a polyethylene glycol derivative and polyoxamers such as methyloxirane (Pluronic L44) or a mixture thereof. Polyethylene glycol with a molecular weight of 106 to 10,000 Da, especially 300 to 6000 Da is preferred.

The polyacrylic acid used in the composition according to the invention in particular has a molecular weight of 1 to 10,000 kDa. The polyacrylic acid may be in linear or crosslinked form.

As a special embodiment, the composition according to the invention comprises curcumin, a solvent, polyacrylic acid and optionally additional excipients, where the polyacrylic acid has a molecular weight of 1 to 10,000 kDa and is optionally crosslinked.

The composition may contain additional excipients such as those known for pharmaceutical or cosmetic compositions. These may include for example, water, neutralizing agents such as NaOH, KOH, trometamol, triethanolamine or diisopropanolamine, thickening agents such as poloxamers, cellulose derivatives or alginates, taste correctors such as essential oils or sweeteners, colouring agents such as food dyes, preservatives such as sorbic acid, benzoic acid, chlorhexidine, benzalkonium chloride or parabens and stabilizers.

According to the present invention, the composition may be present in any form that can be used for the formulation according to the invention.

Liquid, gel or semisolid formulations, for example, gels, creams or ointments are preferred for dermal or mucosal application or solid formulations such as suppositories may be used. Alternatively the formulation according to the invention may also be present in the form of suppositories, preferably vaginal or rectal suppositories.

According to another embodiment, the composition according to the invention may also be present as a combination preparation with an analgesic. Analgesics such as those known in the prior art may be used. For example, analgesics such as morphines, fentanyl or methadone may be used as opioid analgesics, or non-opioid analgesics may be used, such as nicotinergic analgesics or acid anti-inflammatory and antipyretic analgesics, for example, salicylic acid derivatives such as acetylsalicylic acid, phenylacetic acid derivatives such as diclofenac, 2-phenylpropionic acid derivatives such as ibuprofen and naproxen; oxicams such as meloxicam or piroxicam, non-acidic analgesics such as 4-aminophenol derivatives, for example, paracetamol, pyrazolones such as metamizole or phenazone or other non-opioid analgesics such as flupirtine.

In addition, the invention also includes the use of the composition in combination with an analgesic.

For the solid formulations, liquid PEGs or other solvents may be replaced by PEGs or other solvents that are solid and/or semisolid at room temperature, for example. When using PEG, the molecular weight is preferably in the range of 106 to 10,000 Da and in particular between 300 and 6000 Da. Since high-molecular PEGs are liquefied by heating to body temperature and/or on coming in contact with an aqueous medium, the invention disclosed here can also applied to those cases accordingly.

Liquid formulations may be rinses which can be used orally, nasally, vaginally or rectally. Mouth rinses are on specific embodiment. The formulation according to the invention may also be used for a nasal spray or nasal rinse.

The composition according to the invention preferably results in at least a 10% delay, preferably at least a 20% delay in the release of curcumin in the liquid form of the formulation within 30 minutes.

In particular the composition according to the invention is a slow-release liquid formulation.

The invention also includes a pharmaceutical preparation containing the formulation according to the invention and its therapeutic use.

This composition may be used to produce a medication for prevention or treatment of microbial infections, inflammatory diseases or for treatment of cancer or secondary diseases and syndromes caused by cancer or cancer therapies, for example, mucosal inflammations and infections of the mucous membranes in the mouth and throat area as well as the digestive tract.

### EXAMPLES

The following examples are given merely as exemplary and can illustrate better the invention disclosed here. Changes and variations of the following examples are possible within the scope of the present patent claims.

### Example 1

### Solubility Studies with Curcumin

For oral solutions, curcumin must be dissolved. The solvents listed in Table 1 have been tested for this purpose. The maximum concentration of curcumin that could be dissolved is listed for each solvent. If curcumin was soluble, water was added to the solvents and the solubility in this mixture was also determined:

TABLE 1
Solubility of curcumin in organic solvents

| | | Organic solvent + | |
|---|---|---|---|
| No. | Solvent | water mixtures | % Solubility (w/v) |
| 1 | Dimethylacetamide | | 4 |
| 2 | Labrafil | | 3 |
| 3 | N-Ethyl-2-pyrrolidone | 6 + 4 | 4 |
| 4 | N-Ethyl-2-pyrrolidone | 7 + 3 | 5 |
| 6 | N-Ethyl-2-pyrrolidone | | 30 |
| 7 | N-Methyl-2-pyrrolidone | 6 + 4 | 4 |
| 8 | N-Methyl-2-pyrrolidone | 7 + 3 | 4 |
| 10 | N-Methyl-2-pyrrolidone | | 30 |
| 11 | PEG 300 | | 2.5 |
| 12 | PEG 300 | 9 + 1 | 2.5 |
| 13 | PEG 300 | 8 + 2 | 2.5 |
| 14 | Pluronic L44 | | 3 |
| 15 | Pyrrolidone | 1 + 9 | 1 |
| 16 | Pyrrolidone | 5 + 5 | 2.5 |
| 17 | Pyrrolidone | 6 + 4 | 3 |
| 18 | Pyrrolidone | 7 + 3 | 5 |
| 19 | Pyrrolidone | 9 + 1 | 5 |
| 20 | Pyrrolidone | | 20 |
| 21 | Capmul | | 0 |
| 22 | Refined corn oil | | 0 |
| 23 | Cremophor | | 0 |
| 24 | Glycerol | | 0 |
| 25 | Collidone12 | 5 + 5 | 0 |
| 26 | Collidone 12 | 4 + 6 | 0 |
| 27 | Collidone 17 | 5 + 5 | 0 |
| 28 | Collidone 17 | 4 + 6 | 0 |
| 29 | Labrasol | | 0 |
| 30 | Hydrogenated peanut oil | | 0 |
| 31 | Pluronic F-68 | 3 + 7 | 0 |
| 32 | Propylene glycol | | 0 |
| 33 | PVA | | 0 |
| 34 | PVP | 3 + 7 | 0 |

### Example 2

### Preparation of Liquid Curcumin Formulations

Curcumin (0.1 to 2 g) and 0.01-1 g Carbopol 974 NF were dissolved and/or suspended in 100 ml PEG 300. Optionally 1-20% of the PEG 300 component can be replaced by water. Furthermore, polyacrylic acid can be partially or completely neutralized by adding bases such as NaOH, KOH or trometamol. The solutions are dark yellow to brown and have a fine spicy aroma. They have a slightly bitter taste but there is no irritation of the oral mucosa or the gingiva. Mouthwashes with a comparatively higher Carbopol content taste slightly acidic. In general, there is no discoloration of the teeth although the tongue shows an intense yellow colour, but that disappears again after a few hours or after eating. Because of the pleasant taste of the mouthwashes, it is possible to omit the extra additives which influence the taste.

### Example 3

### Storage Stability Studies with Liquid Curcumin Formulations

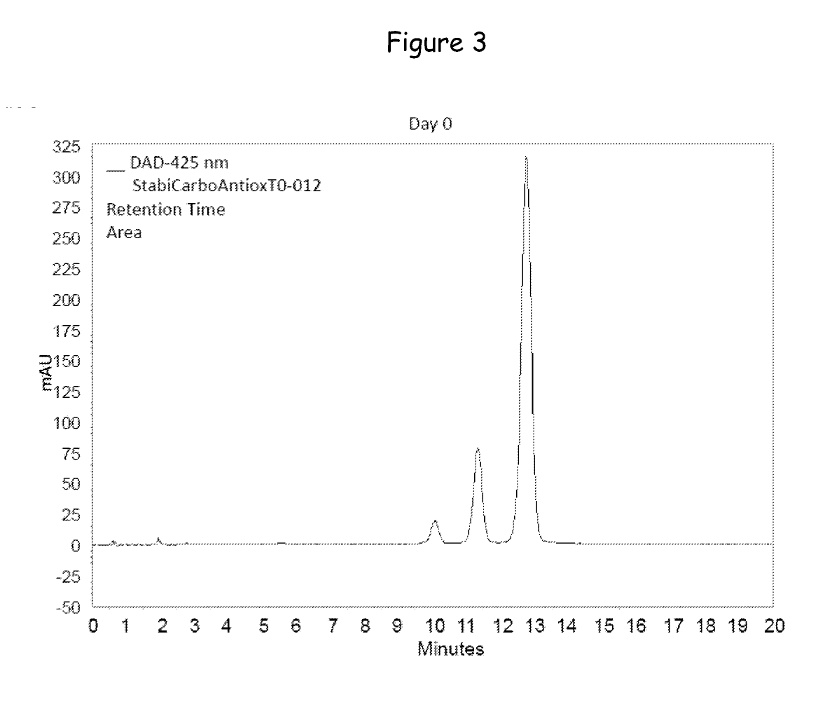

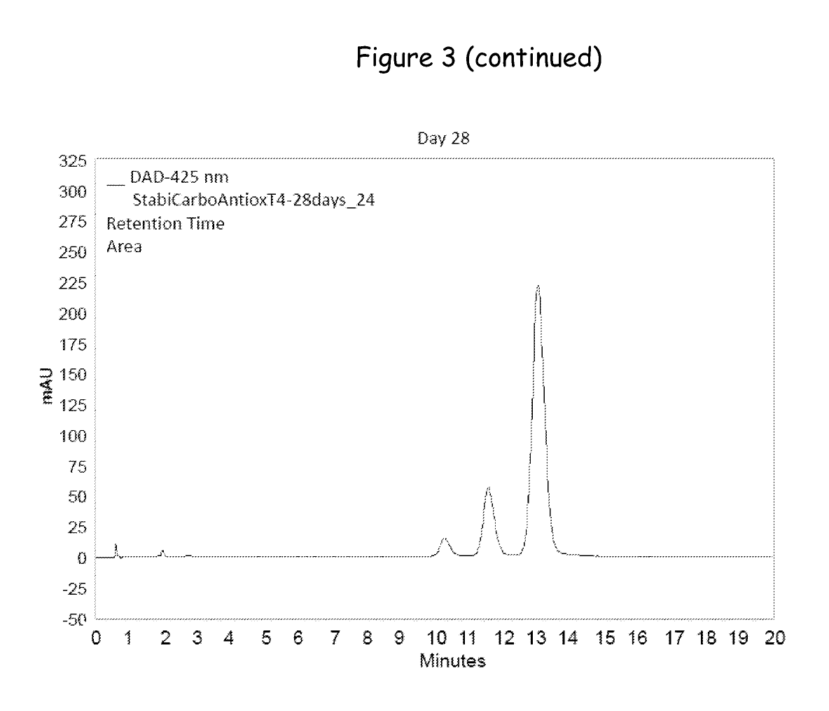

Storage stability studies of the anhydrous solution described in Example 2 under accelerated conditions (40° C.; relative atmospheric humidity 75%) have not revealed any oxidation or degradation of curcumin even after a month. FIG. 3 shows the HPLC spectrum before (top) and after (bottom) one month of storage.

### Example 4

### Preparation of Semisolid Curcumin Formulations

Curcumin ointments were prepared on the basis of PEG using polyethylene glycols in various concentrations and combinations together with polyacrylic acid (Carbopol 974NF). Tables 2 and 3 give a detailed list of the ointments produced.

TABLE 2
Curcumin ointments prepared using different concentrations of
PEG 6000, PEG 600 and PEG 300 as well as polyacrylic acid

| Gel | % PEG | % PEG | % PEG | Carbopol | | |
|---|---|---|---|---|---|---|
| number | 6000 | 600 | 300 | 974 NF | % Curcumin | Total |
| 1 | 12.5 | 19 | 66.5 | 0 | 2 | 100 |
| 2 | 10 | 0 | 87.9 | 0.1 | 2 | 100 |
| 3 | 8 | 0 | 89.9 | 0.1 | 2 | 100 |

TABLE 3
Curcumin ointments prepared using different concentrations of
PEG 4000, PEG 1000 and PEG 400 as well as polyacrylic acid

| Gel | % PEG | % PEG | % PEG | Carbopol | | |
|---|---|---|---|---|---|---|
| number | 4000 | 1000 | 400 | 974 NF | % Curcumin | Total |
| 4 | 5 | 20 | 72.8 | 0.2 | 2 | 100 |
| 5 | 7 | 18 | 72.8 | 0.2 | 2 | 100 |
| 6 | 9 | 16 | 72.8 | 0.2 | 2 | 100 |

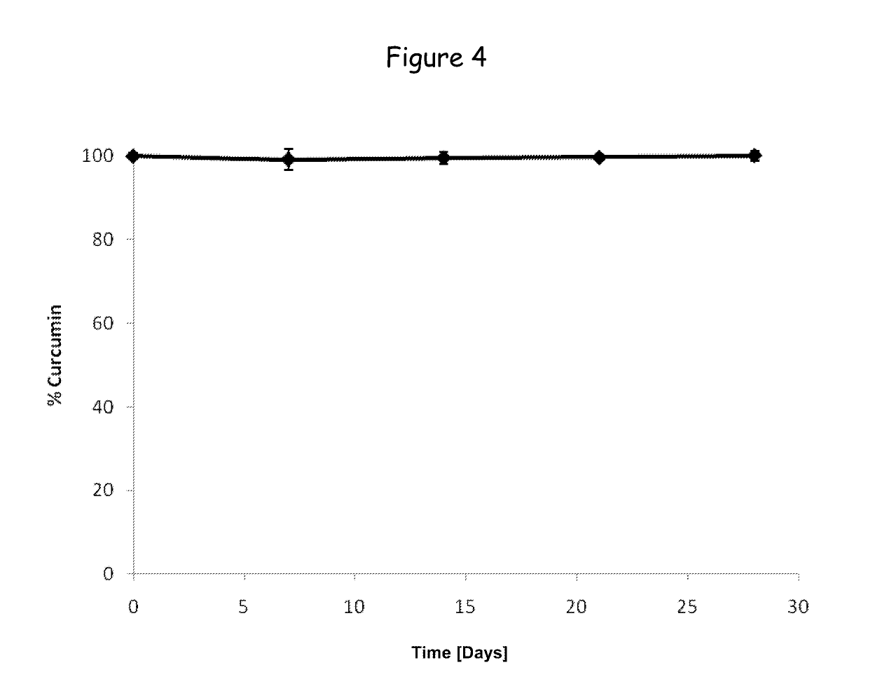

All the ointments had a reddish brown colour and a fine spicy aroma. When used, they cause an intense yellow colour of the skin but without irritation. The colour can easily be washed away with soap and water. Ointment number 1 prepared from 2% curcumin, 12.5% PEG 6000, 19% PEG 600 and 66.5% PEG 300 was stable when stored for four weeks at 40° C./75% relative atmospheric humidity, as shown in FIG. 4. The values that are given correspond to the average of at least three experiments each (±standard deviation). Furthermore, the ointments were stable even after sterilization by autoclaving for 15 minutes.

### Example 5

### Preparation of Solid Curcumin Formulations

Curcumin suppositories for vaginal and/or rectal administration were prepared on the basis of PEG and polyacrylic acid. To prevent the suppositories from liquefying too rapidly at body temperature, poloxamer 408 (=Pluronic F-127) was added. Table 4 shows the precise formulation.

TABLE 4
Formulation for curcumin suppositories

| % | Component |
|---|---|
| 2 | Curcumin |
| 43 | PEG 1000 |
| 24.5 | PEG 4000 |
| 0.5 | Carbopol 974 NF |
| 30 | Pluronic F-127 |

The suppositories did not deform at room temperature. They had an orange-brown colour. Curcumin suppositories were stable at room temperature for a period of three weeks.

### Example 6

### Slow-Release Effect of Polyacrylic Acid

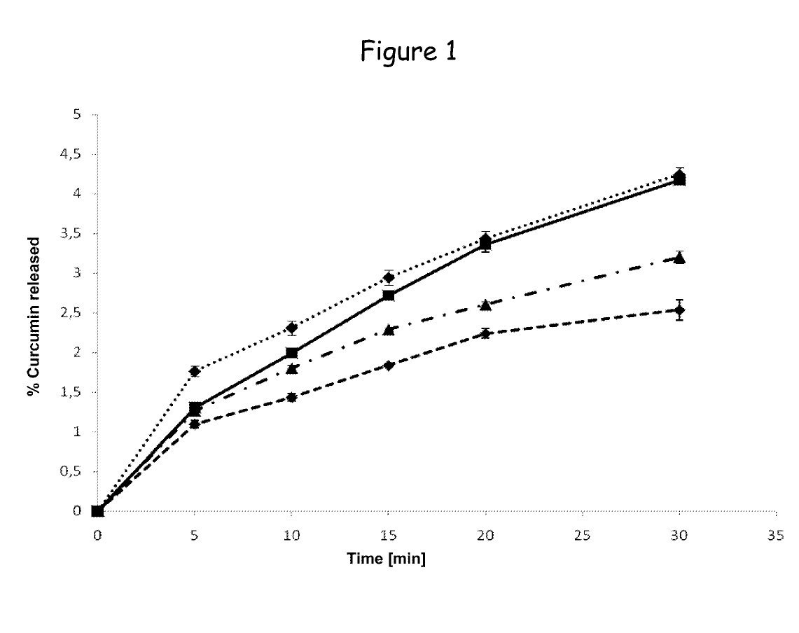

10 ml curcumin solution consisting of 2% (w/v) curcumin dissolved in PEG 300 and various concentrations of Carbopol 974 NF were poured into a dialysis tube, which was then sealed with clamps. A glass beaker was filled with 500 mL of the same solvent as that used for the curcumin solution, and the filled dialysis tube was placed in the glass beaker. The solvent in the glass beaker was cautiously stirred continuously. After 0, 5, 10, 15, 20 and 30 minutes, samples were taken and the extinction at 400 nm was measured using a “FLUOStar OPTIMA Microplate Reader.” The results of this study are shown in FIG. 1 (grey: 0% Carbopol; black: 0.05% Carbopol; grey hatching: 0.1% Carbopol; black hatching: 0.5% Carbopol). It was found that the greater the amount of polyacrylic acid added to the liquid formulation, the more slowly the active ingredient is released from the formulation.

This phenomenon might be based on the development of hydrogen bridge bonds between the phenolic partial structures of curcumin and the carboxylic acid partial structures of polyacrylic acid, which are adequately stabilized by PEG. Although it is known that PEGs are incompatible with phenolic compounds in higher concentrations, this effect was not observed with the combined use of polyacrylic acid with PEG. Despite the binding of curcumin to polyacrylic acid, however, it was found that the polymer surprisingly does not lose its (muco)adhesive properties as a result.

### Example 7

### Mucoadhesive Properties of Curcumin Formulations

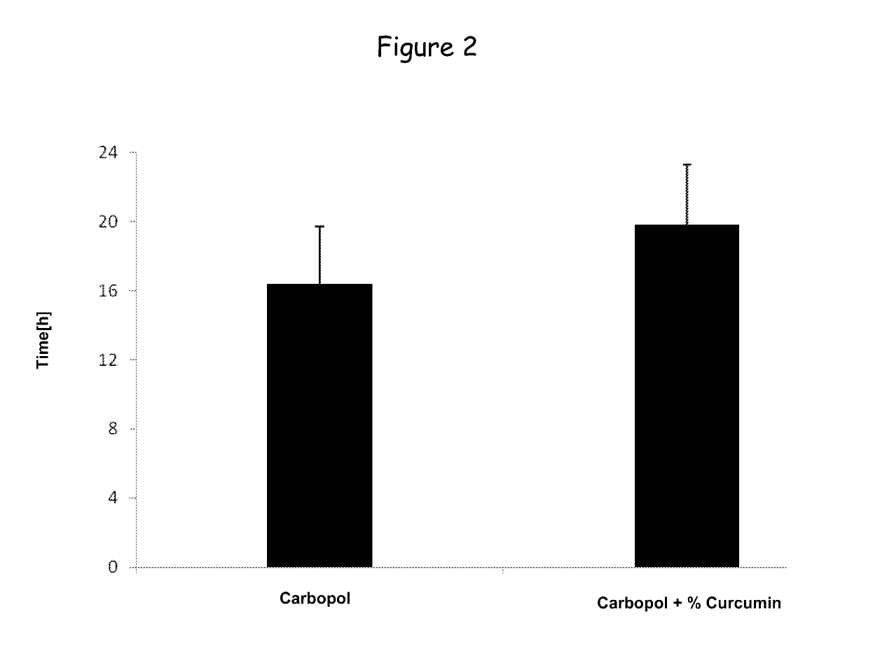

The influence of curcumin on the mucoadhesive properties of polyacrylic acid was tested using the rotating cylinder method (V. Grabovac, D. Guggi, A. Bernkop-Schnürch, Comparison of the mucoadhesive properties of various polymers, Adv. Drug Deliv Rev. 2005 Nov. 3; 57(11):1713-23). The mucoadhesive properties of pure Carbopol 71G NF were compared with those of Carbopol 71G NF containing 2% curcumin. This was done by pressing 30 mg Carbopol with or without 2% curcumin to form test disks with a diameter of 5.0 mm each at a constant pressure. The test disks were placed on fresh bovine buccal mucosa. The mucosa had first been bonded to a stainless steel cylinder (diameter 4.4 cm; height 5.1 cm) using cyanoacrylate adhesive. The cylinder was placed in the dissolution tester (Erweka DT600) containing 0.1 M saline phosphate buffer, pH 7.2, at 37° C.±1° C. The completely immersed cylinder was rotated at 125 revolutions per minute. The adhesion of the test disks was tested after 15, 30, 45, 60, 90, 120, 150 and 180 minutes as well as after 4, 6, 8, 23 and 24 hours. The results of the adhesion testing are plotted graphically in FIG. 2. It was found that an improvement in adhesion was also achieved by adding curcumin.

In addition, it was found that curcumin, which is very unstable in liquid preparations because of its oxidation susceptibility, has a comparatively high stability in storage in PEG formulations.

1. A slow-release formulation containing curcumin, a solvent, polyacrylic acid, a neutralizing agent and optionally additional additives, wherein the polyacrylic acid has a molecular weight of 1 to 10,000 kDa.

2. The slow-release formulation of claim 1, characterized in that curcumin is used in a concentration of 0.1% to 15%.

3. The slow-release formulation of claim 1, characterized in that the solvent is selected from the group consisting of dimethylacetamide, Labrafil, pyrrolidone, N-ethyl-2-pyrrolidone, N-methyl-2-pyrrolidone, polyethylene glycol, polyoxamers and Pluronic L44.

4. The slow-release formulation of claim 1, characterized in that the polyethylene glycol has a molecular weight of 106 Da to 10,000 Da.

5. The slow-release formulation of claim 1, characterized in that the additional excipients are selected from the group consisting of water, neutralizing agents, thickening agents, taste correctors, colouring agents, preservatives and stabilizers.

6. The slow-release formulation of claim 1, characterized in that it the formulation is a liquid or semisolid formulation.

7. The slow-release formulation of claim 1, characterized in that it the formulation is in the form of a mouthwash.

8. The slow-release formulation of claim 1, characterized in that it the formulation is in the form of a gel or an ointment.

9. The slow-release formulation of claim 1, characterized in that it the formulation is a solid.

10. The slow-release formulation of claim 1, characterized in that the formulation is in the form of a suppository.

11. The slow-release formulation of claim 1, characterized in that the formulation is a pharmaceutical or cosmetic preparation.

12. A method of preventing or treating a condition selected from the group consisting of a microbial infection, an inflammatory disease, cancer, and a secondary disease or symptom caused by cancer therapies comprising the step of administering to a subject in need thereof a composition comprising the formulation of claim 1.

13. The slow-release formulation of claim 1, wherein the polyacrylic acid is crosslinked.

14. The slow-release formulation of claim 2, characterized in that curcumin is used in a concentration of 1% to 10%.

15. The slow-release formulation of claim 4, characterized in that the polyethylene glycol has a molecular weight of 300 Da to 6000 Da.

16. The slow-release formulation of claim 10, characterized in that the suppository is a vaginal suppository.
